(19) United States
(12) Patent Application Publication
(10) Pub. No.: US 2010/0317604 A1
(43) Pub. Date: Dec. 16, 2010

(54) FORSYTHIASIDE INJECTION PREPARATION AND PREPARATIVE METHOD THEREOF

(75) Inventors: Zhenyu Xuan, Shanghai City (CN); Yong Wang, Shanghai City (CN); Xiaochun Huang, Shanghai City (CN); Hongguo Lu, Taizhou City (CN); Tianjiang Sun, Taizhou City (CN); Guojun Chen, Taizhou City (CN); Qiuling Tang, Taizhou City (CN)

Correspondence Address: BIRCH STEWART KOLASCH & BIRCH, PO BOX 747, FALLS CHURCH, VA, 22040-0747, US

(73) Assignees: Shanghai Youseen New Medicine Exploition Co., Ltd; Yangtze River Pharmaceutical Group Co., Ltd

(21) Appl. No.: 12/521,494

(22) PCT Filed: Dec. 24, 2007
(86) PCT No.: PCT/CN07/03757

§ 371 Date: Jun. 26, 2009

(30) Foreign Application Priority Data
Dec. 28, 2006 (CN) 200610148180.3

(51) Int. Cl. A61K 31/7034; A61P 31/00

(52) U.S. Cl. 514/25

(57) ABSTRACT

The present invention discloses a Forsythiaside injection preparation and preparative method thereof. This injection preparation is prepared by Forsythiaside and pharmaceutical adjuvant.

### TECHNICAL FIELD

The present invention relates to the domain of pharmaceutical techniques. In detail, it is an injection preparation made of Forsythiaside—an active ingredient extracted from Forsythia Suspensa (Thunb.) The present invention also relates to the preparative method of the Forsythiaside injection preparation.

### BACKGROUND TECHNOLOGY

Forsythiaside is an active ingredient extracted from Forsythia Suspensa (Thunb.) which is a plant of Forsythia from Oleaceae. In the past, we took Forsythin as the indicator of extracting techniques. However, with the deepening research in the active ingredients of Forsythia Suspensa (Thunb.), it has been found that the substance which does boast antibacterial activities is Forsythiaside rather than Forsythin. Forsythiaside plays the main role in exerting the antibacterial activities. The structural formula of Forsythiaside (A) is as follows;

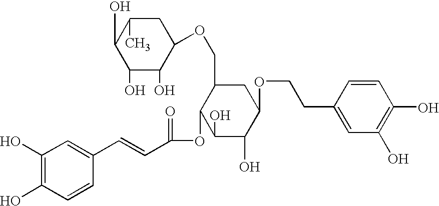

The Forsythiaside referred in documents is a derivative of caffeic acid. There are an ester bond and a glucosidic bond in its molecular structure. It can be easily decomposed under the circumstances of acid, alkali and high temperature. And the caffeic acid, D-glucose, L-rhannose and first glycoside, which are generated after decomposition, have a weakened antibacterial activity. Due to no attention was paid to that problem before, such medicines like Yinqiao Jiedu Pill and Yinqiao Jiedu Tablet can only be detected the ingredient of caffeic acid while the Forsythiaside may have a hydrolysis when processed.

After repeated researches, the inventors believed if people can extract Forsythiaside maximally and get the highly purified active ingredients of it, then such injection preparation can be supplied to pre-existing clinical drugs. In previous technical researches, the inventors have acquired the techniques of extracting highly purified Forsythiaside. However, some pharmaceutical techniques involved in the Forsythiaside injection preparation, such as the type or the dosage of the adjuvant, are not available in the existing technology. Meanwhile, the existing techniques reveal the instability of Forsythiaside which is a serious obstacle in making Forsythiaside into an injection preparation, and there is no inspiration for solving this problem with the existing techniques, besides. Nevertheless, after large amounts of scientific researches, the inventors of this patent have carried out beneficial exploration to make Forsythiaside into an injection preparation feasible and have acquired unexpected results.

### Invention Content:

In view of the above existing disadvantages in techniques, one of the problems to be solved in this invention is to supply a Forsythiaside injection preparation.

Another technical problem to be solved in this invention is to supply the preparative method of this Forsythiaside injection preparation.

This invention is achieved through the following technical schemes:

The Forsythiaside injection preparation mentioned in this invention is mainly prepared by Forsythiaside and pharmaceutical adjuvant with the proportion by weight of 1:(0-5) and the proportion of 1:(0-3) is better, especially.

The Forsythiaside injection preparation involved in this invention has three dosage forms: namely, lyophilized powder injection, water injection and infusion solution.

The Forsythiaside mentioned in this invention is the one whose purity no less than 90% or the one which can be applied in the injection. It can be extracted from the plant of Forsythia or we can get it by purchasing or synthesis.

The pharmaceutical adjuvant used in the Forsythiaside lyophilized powder injection in present invention is called frame agent which is a compound containing one or two compositions among which contains one or a mixture of any two of the following Mannitol, Glucose and Sorbitol randomly. The optimizing technical scheme is to add no frame agent into the Forsythiaside lyophilized powder injection. That means the proportion of Forsythiaside to frame agent is 1:0 by weight.

The Forsythiaside lyophilized powder injection in this invention is prepared through the following measures: mixing Forsythiaside and frame agent by their proportion of weight, and adding water for injection to it for dissolution with its weight 10-50 times of the weight of the Forsythiaside, then adjusting the pH value to 3.0-6.0, filling it into ampoules after refined filtration and ultrafiltration. And the lyophilized injection is finally accomplished after freezing and drying referring to the Table 1—Lyophilizing Curve.

TABLE 1
Lyophilizing Curve of The Forsythiaside Lyophilized Powder Injection

| Temperature (° C.) | Time (h) |
|---|---|
| 0-−40 | 2 |
| −40 | 4 |
| −40-−25 | 3 |
| −25 | 5 |
| −25-30 | 2 |
| −30 | 9 |

The pharmaceutical adjuvant used in the Forsythiaside water injection is called stabilizing agent which contains one or several kinds of the following Disodium EDTA, Sodium-Calcium EDTA, Calcium EDTA, Vitamin C and Pyrosulfite.

The Forsythiaside water injection in this invention is prepared through the following measures: adding the stabilizing agent to water for injection with its weight 0 to 5 times the weight of the Forsythiaside, then stirring the solution until it dissolves completely, adding activated carbon with 0.5-0.05% weight of the total amount, stirring, filtering and decarbonizing, then adding Forsythiaside to the filtrate and making it fully dissolved, regulating the pH value to 3.0-6.0, adding activated carbon again with 0.2-0.02% weight of the total amount, stirring the solution at room temperature, filtering, decarbonizing, and adding water for injection to the filtrate for filling up to the total amount. Measuring the pH value and the active constituent content, then. After being qualified, the injection should be filtered repeatedly to being clear, then filling it separately into ampoules, and have a sterilization and a package later.

The pharmaceutical adjuvant used in the Forsythiaside infusion solution is called stabilizing agent which contains one or several kinds of the following Disodium EDTA, Sodium-Calcium EDTA, Calcium EDTA, Vitamin C and Pyrosulfite.

The Forsythiaside infusion solution is prepared through the following measures: adding sodium chloride or glucose to water for injection and adding the stabilizing agent to it with its weight 0 to 5 times of the weight of the Forthiaside, dissolving it fully. Adding activated carbon with 0.5-0.05% weight of the total amount, stirring, filtering and decarbonizing, then adding Forsythiaside to the filtrate and making it fully dissolved by stirring, regulating the pH value to 3.0-6.0, adding activated carbon again with 0.2-0.02% weight of the total amount, stirring the solution at room temperature, filtering, decarbonizing, and adding water for injection to the filtrate for filling up to the total amount, measuring the pH value and the active constituent content. After being qualified, the solution should be filtered repeatedly to being clear, then filling it separately into ampoules, and have a sterilization and a package later.

In the preparation for the water injection and infusion solution mentioned above, the adding amount of the activated carbon is a proportion comparing to the total volume.

These above technical schemes applied in present invention have the following advantages:
1. It is a creation to use Forsythiaside as an active ingredient in the injection preparation. Forsythiaside has always been a hot spot in research due to its role in antibiosis and anti-virus. What's more, Forsythiaside is considered as a detection index for medicine's constituents, yet which has not been achieved. And there haven't the reports on taking Forsythiaside as the active ingredient in the injection preparation with current techniques yet. And the relative pharmaceutical techniques on the Forsythiaside injection preparation, such as the category or the dosage of adjuvant, are also not available. Besides, there doesn't exist the relative inspiration for taking Forsythiaside as the active ingredient in the injection preparation. However, the present invention has determined the category and the dosage of the pharmaceutical adjuvant and the lyophilizing art of the lyophilized powder injection after strict experimental screening.
   The present invention has also overcome the technical bias: The regular lyophilized powder injection can take shape only if some protective adjuvant were added during the preparation. While the present invention indicates that the Forsythiaside lyophilized powder injection can take shape well only by freezing it directly. Besides, it is easy to be frozen without adding adjuvant. It is also an unexpected result in techniques that the Forsythiaside lyophilized powder injection can meet the demand of the injection preparation without adding any pharmaceutical adjuvant. That not only realizes the simplicity of the techniques, save the cost but also avoids the insecurity problem brought by adjuvant.
   The present invention is invented according to the feature of Forsythiaside. As the Forsythiaside is unstable at a high temperature, the whole techniques applied in present invention are under freezing conditions or at low temperatures to avoid the active ingredient of Forsythiaside being destroyed. The powder of the lyophilized powder injection has a loose texture and a good re-dissolving effect after adding water to it. And the infusion solution and the water injection can also have a fine stability with the use of adjuvant.
2. The present invention can be implemented in the industrial production. Meanwhile this invention has an excellent treatment effect, especially in the fields of fever relief and anti-inflammation. The present invention also takes effect on various bacteria and virus that undoubtedly means it boasts promising markets and social benefits. So this invention has rather utility.

### ILLUSTRATIONS

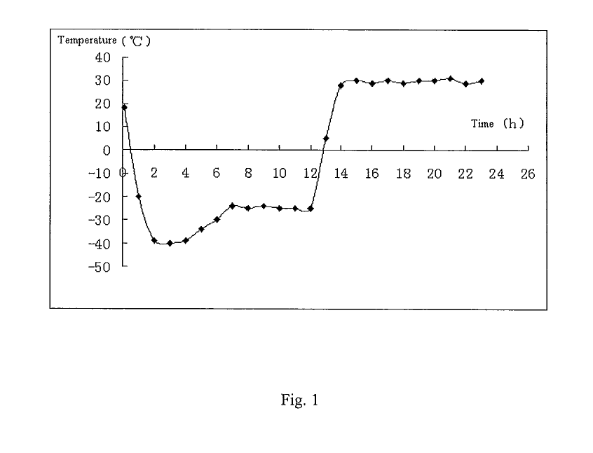

FIG. 1 is a curve table depicting the lyophilizing artwork of the Forsythiaside lyophilized powder injection of this invention.

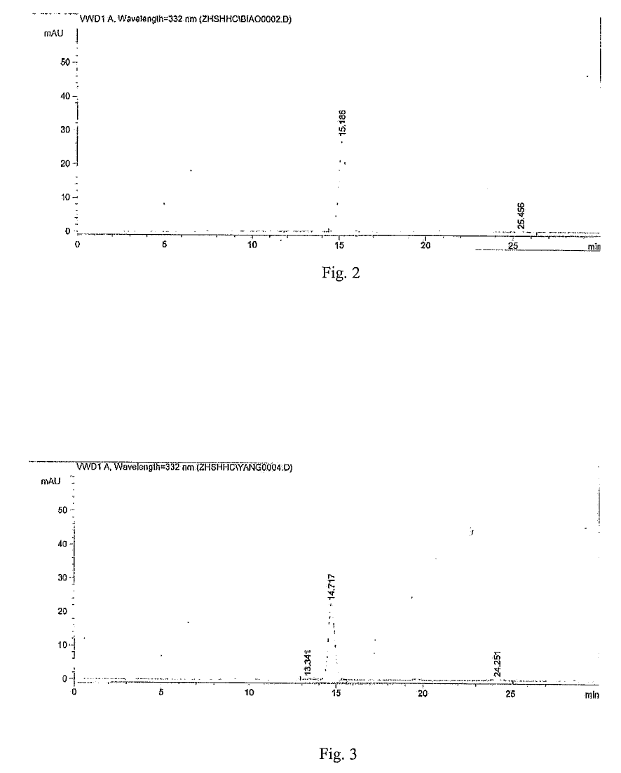

FIG. 2 is a liquid chromatogram of the standard reference material of Forsythiaside in present invention.

FIG. 3 is a liquid chromatogram of the sample product of the Forsythiaside lyophilized powder injection of this invention.

### THE BEST PRACTICAL METHOD

To further explain the present invention, the following practical examples are given, in which: example 1 to 9 are about lyophilized powder injection, example 10 to 14 are about water injection and example 15 to 18 are about infusion solution.

### Example 1

Taking 75 g Forsythiaside, adding 1200 ml water for injection, stirring and dissolving it, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them referring to the lyophilizing curve before the preparation is accomplished. The liquid chromatogram of the standard reference material of Forsythiaside is as shown in FIG. 2. And the liquid chromatogram of the Forsythiaside lyophilized powder injection which is prepared through this method is as shown in FIG. 3.

The lyophilizing curve is as shown in Table 1 followed:

| Temperature (° C.) | Time (h) |
|---|---|
| 0-−40 | 2 |
| −40 | 4 |
| −40-−25 | 3 |
| −25 | 5 |
| −25-30 | 2 |
| −30 | 9 |

### Example 2

Taking 75 g Forsythiaside and Mannitol, respectively. Mixing them, adding 2000 ml water for injection to it, then stirring and dissolving it. Regulating the pH value to 5.5 with Acetic Acid-Sodium Acetate buffer salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 3

Taking 50 g Forsythiaside and 100 g Glucose. Mixing them, adding 1000 ml water for injection to it, then stirring and dissolving it. Adjusting the pH value to 5.0-6.0 with Citric Acid-Sodium Dihydrogen Phosphate buffet salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 4

Taking 100 g Forsythiaside, 75 g Mannitol and Glucose, respectively. Mixing them, adding 2500 ml water for injection to it, then stirring and dissolving it. Adjusting the pH value to 3.0 with Acetate buffer salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 5

Taking 100 g Forsythiaside, Mannitol and Sorbitol separately. Mixing them, adding 3000 ml water for injection to it, then stirring and dissolving it. Adjusting the pH value to 6.0 with Citric Acid-Sodium Dihydrogen Phosphate buffer salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 6

Taking 150 g Forsythiaside and Glucose, respectively. Mixing them, adding 5000 ml water for injection to it, then stirring and dissolving it. Adjusting the pH value to 4.5 with Acetic Acid-Sodium Acetate buffer salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 7

Taking 150 g Forsythiaside, Glucose and Sorbitol separately. Mixing them, adding 6000 ml water for injection to it, then stirring and dissolving it. Adjusting the pH value to 5.5 with Citric Acid-Sodium Dihydrogen Phosphate buffet salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 8

Taking 200 g Forsythiaside and 600 g Sorbitol. Mixing them, adding 8000 ml water for injection to it, then stirring and dissolving it. Adjusting the pH value to 6.0 with Citric Acid-Sodium Dihydrogen Phosphate buffet salt, filtering it with 0.22 μm microporous membrane for sterilization, then depyrogenating it with 6000 Dal ultrafiltration membrane. After the intermediate's assay is approved, filling the injection up to the fixed volume with water for injection and filling it separately into 4000 ampoules. Putting them into the refrigerator and freezing them according to the lyophilizing curve before the preparation is accomplished. The lyophilizing curve applied in this example is the same as the one in Example 1.

### Example 9

Taking 75 g Forsythiaside, 200 g Mannitol and 175 g Glucose. Mixing them, adding 3750 ml water for injection to it, then stirring and dissolving it. Adjusting the pH value to 6.0 with Citric Acid-Sodium Dihydrogen Phosphate buffet salt, filtering it with 0.22 μm microporous membrane for sterilization, then depyrogenating it with 8000 Dal ultrafiltration membrane. After the intermediate's assay is approved, filling the injection up to the fixed volume with water for injection and filling it separately into 1000 ampoules. Putting them into the refrigerator and freezing them according to the lyophilizing curve before the preparation is accomplished. The lyophilizing curve applied in this example is the same as the one in Example 1.

### Example 10

Adding 2.5 g Sodium-Calcium EDTA to water for injection and dissolving it completely. Adding activated carbon with 0.05% weight of the total amount (2 L), stirring, filtering and decarbonizing, then adding 75 g Forsythiaside to the filtrate and making it fully dissolved. Regulating the pH value to 3.0-6.0 with Citric Acid-Sodium Dihydrogen Phosphate buffet salt. Adding activated carbon again with 0.02% weight of the total amount (2 L), stirring the solution at room temperature, filtering, decarbonizing and filling the filtrate up with water for injection to the total amount of 2 L. Measuring the pH value and the active constituent content, then. After being qualified, the injection should be filtered repeatedly to being clear, then filling it separately into 1000 ampoules, and have a sterilization and a package later.

### Example 11

Adding 2.5 g Pyrosulfite to water for injection and dissolving it completely. Adding activated carbon with 0.5% weight of the total amount(5 L), stirring, filtering and decarbonizing, then adding 150 g Forsythiaside to the filtrate and making it fully dissolved. Regulating the pH value to 3.0-6.0 with Citric Acid-Sodium Dihydrogen Phosphate buffet salt. Adding activated carbon again with 0.2% weight of the total amount(5 L), stirring the solution at room temperature, filtering, decarbonizing and filling the filtrate up with water for injection to the total amount of 5 L. Measuring the pH value and the content of Forsythiaside, then. After being qualified, the injection should be filtered repeatedly to being clear, then filling it separately into 1000 ampoules, and have a sterilization and a package later.

### Example 12

Adding 2.5 g Disodium EDTA to water for injection and dissolving it completely. Adding activated carbon with 0.1% weight of the total amount(10 L), stirring, filtering and decarbonizing, then adding 150 g Forsythiaside to the filtrate and making it fully dissolved. Regulating the pH value to 3.0-6.0 with Citric Acid-Sodium Dihydrogen Phosphate buffet salt. Adding activated carbon again with 0.1% weight of the total amount(10 L), stirring the solution at room temperature, filtering, decarbonizing and filling the filtrate up with water for injection to the total amount of 10 L. Determining the pH value and the content of Forsythiaside, then. After being qualified, the injection should be filtered repeatedly to being clear, then filling it separately into 1000 ampoules, and have a sterilization and a package later.

### Example 13

Adding 225 g Vitamin C to water for injection and dissolving it completely. Adding activated carbon with 0.4% weight of the total amount (5 L), stirring, filtering and decarbonizing, then adding 75 g Forsythiaside to the filtrate and making it fully dissolved. Regulating the pH value to 3.0-6.0 with Citric Acid-Sodium Dihydrogen Phosphate buffet salt. Adding activated carbon again with 0.05% weight of the total amount(5 L), stirring the solution at room temperature, filtering, decarbonizing and filling the filtrate up with water for injection to the total amount of 5 L. Determining the pH value and the content of Forsythiaside, then. After being qualified, the injection should be filtered repeatedly to being clear, then filling it separately into 1000 ampoules, and have a sterilization and a package later.

### Example 14

Adding 375 g Vitamin C to water for injection and dissolving it completely. Adding activated carbon with 0.1% weight of the total amount (10 L), stirring, filtering and decarbonizing, then adding 75 g Forsythiaside to the filtrate and making it fully dissolved. Regulating the pH value to 3.0-6.0 with Citric Acid-Sodium Dihydrogen Phosphate buffet salt. Adding activated carbon again with 0.2% weight of the total amount (10 L). Stirring the solution at room temperature, filtering, decarbonizing and filling the filtrate up with water for injection to the total amount of 10 L. Determining the pH value and the content of Forsythiaside, then. After being qualified, the injection should be filtered repeatedly to being clear, then filling it separately into 1000 ampoules, and have a sterilization and a package later.

### Example 15

Adding 9.0 g Sodium Chloride and 0.1 g Sodium-Calcium EDTA to water for injection and dissolving it completely. Adding activated carbon with 0.05% weight of the total amount(1 L), stirring, filtering and decarbonizing, then adding 0.6 g Forsythiaside to the filtrate and making it fully dissolved. Regulating the pH value to 3.0-6.0 with Citric Acid-Sodium Dihydrogen Phosphate buffet salt. Adding activated carbon again with 0.02% weight of the total amount(1 L), stirring the solution at room temperature, filtering, decarbonizing and filling the filtrate up with water for injection to the total amount of 1 L. Determining the pH value and the content of Forsythiaside, then. After being qualified, the solution should be filtered repeatedly to being clear, then filling it separately into 4 bottles, and have a sterilization and a package later.

### Example 16

Adding 9.0 g Sodium Chloride and 0.1 g Pyrosulfite to water for injection and dissolving it completely. Adding activated carbon with 0.3% weight of the total amount(1 L), stirring, filtering and decarbonizing, then adding 0.3 g Forsythiaside to the filtrate and making it fully dissolved. Adjusting the pH value to 3.0-6.0 with Citric Acid-Sodium Dihydrogen Phosphate buffet salt. Adding activated carbon again with 0.05% weight of the total amount(1 L), stirring the solution at room temperature, filtering, decarbonizing and filling the filtrate up with water for injection to the total amount of 1 L. Determining the pH value and the content of Forsythiaside, then. After being qualified, the solution should be filtered repeatedly to being clear, then filling it separately into 4 bottles, and have a sterilization and a package later.

### Example 17

Adding 50 g Glucose and 0.9 g Vitamin C to water for injection and dissolving it completely. Adding activated carbon with 0.05% weight of the total amount (2 L), stirring, filtering and decarbonizing, then adding 0.3 g Forsythiaside to the filtrate and making it fully dissolved. Adjusting the pH value to 3.0-6.0 with Citric Acid-Sodium Dihydrogen Phosphate buffet salt. Adding activated carbon again with 0.1% weight of the total amount (2 L), stirring the solution at room temperature, filtering, decarbonizing and filling the filtrate up with water for injection to the total amount of 2 L. Determining the pH value and the content of Forsythiaside, then. After being qualified, the solution should be filtered repeatedly to being clear, then filling it separately into 4 bottles, and have a sterilization and a package later.

### Example 18

Adding 100 g Glucose and 1.5 g Vitamin C to water for injection and dissolving it completely. Adding activated carbon with 0.5% weight of the total amount (1 L), stirring, filtering and decarbonizing, then adding 0.3 g Forsythiaside to the filtrate and making it fully dissolved. Adjusting the pH value to 3.0-6.0 with Citric Acid-Sodium Dihydrogen Phosphate buffet salt. Adding activated carbon again with 0.1% weight of the total amount (1 L), stirring the solution at room temperature, filtering, decarbonizing and filling the filtrate up with water for injection to the total amount of 1 L. Determining the pH value and the content of Forsythiaside, then. After being qualified, the solution should be filtered repeatedly to being clear, then filling it separately into 4 bottles, and have a sterilization and a package later.

The detailed explanations of the considerable effects on fever relief and anti-inflammation of the Forsythiaside injection preparation in present invention will be followed up with combinations of the experiments on pharmacodynamics. The pharmacodynamics results of the Forsythiaside infusion solution can be referred to that of the Forsythiaside water injection.

### 1.1 Pharmacodynamics Experiment on Fever-Relief

Taking SD rats 48 in number, dividing them into 4 groups randomly, namely NS control group, SHL (ShuangHuangLian) control group, group of the Forsythiaside lyophilized powder injection, which is prepared according to one of the example 1 to 9, group of the Forsythiaside water injection, which is prepared according to one of the example 10 to 14. Before the experiment, measuring each rat's body temperature twice, and taking the mean values as their basal temperatures. A subcutaneous injection of 1 ml/kg of 1.5 mg/ml extemporized 2,4-Dinitrophenol is administered to the backs of rats, shortly after each group's rats were injected via the tail vein with the corresponding medicines by 10 ml/(kg·d) and 6 times the amount of clinical daily expenses. Measuring and recording animal's temperature at 30, 60, 90, 120, 180 and 240 minutes after taking the medication.

The experiment result shows: in comparison with the NS control group, group of the Forsythiaside lyophilized powder injection shows a significant antipyretic effect on SD rats' fever(p<0.05)30 minutes later after taking the medication. After taking the medication 60-120 minutes, group of the Forsythiaside water injection and group of the Forsythiaside lyophilized powder injection show significant antipyretic effects in varying degrees on SD rats' fever(p<0.05,p<0.01) comparing to the NS control group. The SHL control group shows antipyretic effect on SD rats' fever comparing to the NS control group after taking the medication 30 min-180 min(p<0.01,p<0.05), while it shows no antipyretic effect comparing to the NS control group 240 minutes later after taking the medication. The result obtained is given set forth in Table 2.

TABLE 2
Temperature Changes of the Rats at Different Hours After Taking 2,4-Dinitrophenol in the Experiment on Fever-relief (° C.) ( x̅ ± s)

| | Mean Temperature | Temperature Changes at Different Hours after Medication | | | | | |
|---|---|---|---|---|---|---|---|
| Group | before Medication | 0.5 h | 1 h | 1.5 h | 2 h | 3 h | 4 h |
| NS Group | 38.19 ± 0.25 | 0.77 ± 0.13 | 1.47 ± 0.26 | 1.45 ± 0.26 | 1.37 ± 0.22 | 1.12 ± 0.23 | 0.68 ± 0.25 |
| SHL Control Group | 38.18 ± 0.28 | 0.51 ± 0.26* | 0.81 ± 0.28** | 0.84 ± 0.28** | 0.64 ± 0.32** | 0.59 ± 0.21** | 0.47 ± 0.20 |
| the Forsythiaside | 38.19 ± 0.14 | 0.55 ± 0.11 | 1.05 ± 0.12** | 1.22 ± 0.24* | 1.13 ± 0.26* | 1.05 ± 0.27 | 0.67 ± 0.22 |
| Water Injection | | | | | | | |
| the Forsythiaside | 38.15 ± 0.25 | 0.56 ± 0.22* | 1.02 ± 0.23** | 0.82 ± 0.28** | 0.78 ± 0.28** | 0.56 ± 0.30** | 0.46 ± 0.28 |
| Lyophilized Powder | | | | | | | |
| Injection | | | | | | | |

Note:
In comparison with NS control group,
*P < 0.05,
**P < 0.01

### 1.2 Pharmacodynamics Experiment on Anti-Inflammation

### 1.2.1 The Forsythiaside Injection Preparation's Effects on the Mice's Swelling Ears Caused by Xylene

Taking mice 48 in number, dividing them into 4 groups, the group categories are the same as those in the pharmacodynamics experiment on fever-relief mentioned above. Giving the medication of 10 ml/(kg·d) by injection for 4 days and with the amount equaling to 10 times the amount of clinical daily expenses. 30 minutes later after giving the last medication, smearing 50 μl Xylene to each mouse's left ear and left the right ear as the blank control. Executing the mouse 15 min later after causing inflammation, and cutting both ears, punching down a piece of ear with a diameter of 7 mm from each ear by a puncher. Weighting these two ears slices and calculating the swelling degree and the swelling inhibition ratio.

### Formula:

$$Swelling degree = Weight of the left ear slice □ Weight of the right ear slice$$

$$Swelling inhibition ration = \frac{\begin{matrix}Swelling degree of the blank control group □ \\ Swelling degree of the medication group\end{matrix} }{Swelling degree of the blank control group} \times 100 □$$

The experiment shows: mice in the NS group have red and swollen left ears whose swelling degrees are up to 0.0145±0.0037 g while the swelling degrees of group of each Forsythiaside injection preparation and SHL control group are all lower than those of the NS control group. And all of them have significant anti-inflammation effects on the mice's swellings ears in varying degrees comparing to the NS control group.(p<0.05,p<0.01). The result obtained is given set forth in Table 3.

TABLE 3
The Forsythiaside Injection Preparation's Effects on the Mice's
Swelling Ears Caused by Xylene

| | | | Swelling |
|---|---|---|---|
| | | Swelling | Inhibition |
| Group | Number | Degree ( x̅ ± s, g) | Ration % |
| NS Group | 12 | 0.0145 ± 0.0037 | — |
| SHL control group | 12 | 0.0099 ± 0.0033** | 31.7 |
| the Forsythiaside Water | 12 | 0.0109 ± 0.0028* | 25.8 |
| Injection | | | |
| the Forsythiaside | 12 | 0.0087 ± 0.0039* | 40.0 |
| Lyophilized Powder | | | |
| Injection | | | |

Note□
In comparison with NS control group□
*P < 0.05□
**P < 0.01

### 1.2.2 The Experiment of the Forsythiaside Injection Preparation's Effects on Vasopermeability

Taking 48 mice with a weight ranging from 18 to 22 g in which male mice and female ones are equal in number. The group categories are the same as those in the pharmacodynamics experiment on fever-relief mentioned above. After taking the corresponding medicines 1 h, administrating tail vein injection with 0.5% Evans Blue Normal Saline of 0.1 ml/10 g by weight, then. Giving an intraperitoneal injection with 0.6% Acetic Acid by 0.20 ml per mouse, 20 min later, executing the mice by breaking the cervical vertebra. Then cutting the skin and muscle of abdomen with a scissor, cleaning the abdominal cavity with 6 ml Normal Saline by three times, sucking the solution with a pipette and adding Normal Saline to 10 ml after the combination, centrifuging the combining solution 15 min at the speed of 3000 rpm. Removing the clear solution on the top and having a colorimetric determination at 590 nm, recording the OD value and conducting a statistical analysis.

The experiment result shows: in comparison with the NS control group, group of the Forsythiaside water injection and group of the Forsythiaside lyophilized powder injection have significant effects on vasopermeability(p<0.05), and the SHL control group also has a significant effect on vasopermeability comparing with the NS control group(p<0.05).The result obtained is given set forth in Table 4.

TABLE 4
the Forsythiaside Injection Preparation's Effects on the Improvements
of the Vasopermeability in Mice's Abdominal Cavity after Having an
Intraperitoneal Injection of Acetic Acid

| Group | Number | Weight | OD Value |
|---|---|---|---|
| NS Group | 10 | 21.98 ± 1.14 | 0.0883 ± 0.0208 |
| SHL control group | 10 | 22.05 ± 1.07 | 0.0667 ± 0.0155* |
| the Forsythiaside | 10 | 22.03 ± 1.20 | 0.0690 ± 0.0140* |
| Water Injection | | | |
| the Forsythiaside | 10 | 22.14 ± 0.96 | 0.0685 ± 0.0131* |
| Lyophilized Powder | | | |
| Injection | | | |

Note□
In comparison with NS control group□
*P < 0.05□
**P < 0.01

For the next, we will take the lyophilized powder injection as the example to clearly explain reasons for choosing pharmaceutical adjuvant, the pH value in the preparation and the lyophilizing curve of the Forsythiaside lyophilized powder injection. And we will also specify the stability of the Forsythiaside lyophilized powder injection by accelerated test.

### 2.1 Choice of the Frame Agents

The commonly used frame agents in the lyophilized powder injection are Mannitol, Glucose, Sorbitol, etc. We have designed an experiment for choosing a suitable type of frame agent. The result obtained is given set forth in Table 5.

TABLE 5
Experiment on the Types of Frame Agents in the Forsythiaside
Lyophilized Powder Injection

| Type of | Volume of the | | Appearance of the | |
|---|---|---|---|---|
| the Frame Agent | Replenishment | Redissolution | Product | Dissolubility |
| No frame | 2 ml | Fine | Faint yellow lump, | Fine |
| agent | | | relatively loose | |
| No frame | 1.5 ml | Fine | Faint yellow lump, | Fine |
| agent | | | loose | |
| 10% Mannitol | 1.5 ml | Fine | Faint yellow lump, | Fine |
| | | | relatively tight | |
| 20% Mannitol | 1.5 ml | Fine | Faint yellow lump, | Fine |
| | | | relatively tight | |
| 30% Mannitol | 1.5 ml | Fine | Ivory and tight | Fine |
| | | | lump | |
| 50% Mannitol | 1.5 ml | Fine | Ivory and tight | Fine |
| | | | lump | |
| 5% Glucose | 1.5 ml | Fine | Shrink | Fine |
| 10% Glucose | 1.5 ml | Fine | Shrink | Fine |
| 5% Sorbitol | 1.5 ml | Fine | Ivory and tight | Fine |
| | | | lump | |

TABLE 6
Experiment on the Adding Amounts of Frame Agents
in the Forsythiaside Lyophilized Powder Injection

| | Adding Amounts of the Frame Agents (%) | | | | | | |
|---|---|---|---|---|---|---|---|
| Forsythiaside | 0 | 50 | 100 | 200 | 300 | 400 | 500 |
| Shaping Property | General | General | Fine | Fine | Fine | General | Relatively poor |
| Dissolubility | Fine | Fine | Fine | Fine | Fine | General | General |
| Clarity | Fine | Fine | Fine | General | General | Relatively poor | Relatively poor |

The research on the adding amounts of frame agents shows, when the proportion of Forsythiaside to frame agent is 1:(0-5) by weight, all the lyophilized powder can take shape well. However, the powder's shaping property, the dissolubility and the clarity after redissolution are even better when the weight proportion is 1:0-3. The present invention can obtain the powder which has a good shape, a fine dissolubility and a high clarity by freezing directly without adding any frame agent. And it is also easy for lyophilizing through this way.

### 2.2 Choice of the pH Value

Taking appropriate amount of Forsythiaside, adding water to it for dissolution. Adjusting the pH value to 3.0, 3.5, 4.0, 4.5, 5.0, 5.5, 6.0 separately, then determining the peak area value of Forsythiaside at different pH value by HPLC to investigate the influence on the content of Forsythiaside caused by different pH value.

The result shows that: when the pH value is 4.5, Forsythiaside has the highest content and its content is not sufficiently varied when pH value is at 3.0-6.0. The result obtained is given set forth in Table 7. So the pH value of the Forsythiaside lyophilized powder injection in present invention is controlled at 3.0-6.0.

TABLE 7
Result of the Experiment on Choice of The pH Value

| | pH Value | | | | | | |
|---|---|---|---|---|---|---|---|
| | 3.0 | 3.5 | 4.0 | 4.5 | 5.0 | 5.5 | 6.0 |
| Peak | 1410.0 | 1499.0 | 1519.8 | 1523.1 | 1494.5 | 1480.6 | 1423.5 |
| Area | | | | | | | |

### 2.3 Choice of the Lyophilized Artwork Curve

### 2.3.1 Setting Freezing Temperature

Generally, the temperature of the shelf should be 5° C.-15° C. lower than the eutectic point of the products. According to the results which have determined above, the eutectic point of the product in this invention is −20° C., so the freezing temperature should be under −35° C. and we set −40° C. as the freezing temperature after selection.

### 2.3.2 Setting Freezing Time

In present invention each filling amount of the lyophilized powder injection is 1.5 ml. After the experiment, we find that, it takes about 4 h for the lyophilized powder injection in present invention to be frozen totally under the temperature of −40° C.

### 2.3.3 Sublimation Velocity and Drying Time

Experiment shows: when under the temperature of −25° C., having a drying time of 5 hours in the first phase and another 9 hours' drying in the second phase when the temperature is under 30° C., the demand of the moisture content in lyophilized powder injection can be met.

Prepare 3 batches of lyophilized powder injection according to the above screened optimal techniques, filling 1.5 ml into each ampoule for lyophilizing. The preliminarily screened lyophilizing artwork curve refers to FIG. 1.

Lyophilize 10 batches of finished products according to the preliminarily screened lyophilizing artwork curve, and detect the lyophilization-related data e.g. moisture etc. The detection result refers to Tab.8. The result shows all finished products meet the criterion and proves that the curve is feasible.

TABLE 8
Observation on lyophilizing result for finished products

| Finished Products | | Moisture | Forsythiaside |
|---|---|---|---|
| Lot Number | Description | Content (%) | Content (%) |
| 2006060601 | meet the criterion | 3.24 | 92.00 |
| 2006060602 | meet the criterion | 3.68 | 92.05 |
| 2006060603 | meet the criterion | 3.71 | 91.89 |
| 2006060801 | meet the criterion | 3.15 | 91.93 |
| 2006060802 | meet the criterion | 3.57 | 92.08 |
| 2006060803 | meet the criterion | 3.81 | 91.92 |
| 2006060804 | meet the criterion | 3.09 | 92.31 |
| 2006061101 | meet the criterion | 3.60 | 91.71 |
| 2006061102 | meet the criterion | 3.25 | 92.15 |
| 2006061103 | meet the criterion | 2.91 | 92.07 |

### 2.4 Accelerated Testing

Through accelerating medicine's chemical or physical changes, can we approach the stability of a pharmaceutical preparation. And it also provides necessary information in the fields of prescription design, technique improvement, quality research, package improvement, transportation and storage. The conditions that need to be observed are as follows:
Package: The package which is intended to be sold on the market (Penicillin Bottle+Butyl Rubber Plugs)
Temperature: 40° C.±2° C.
Humidity: 75%±15%

TABLE 9
Result of the Accelerated Test

| | | Accelerated Test | | | | |
|---|---|---|---|---|---|---|
| Dosage Form | | 0 day | 15 days | 1 month | 2 months | 3 months |
| Lyophilized | Description | Solid powder | Solid powder | Solid powder | Solid powder | Solid powder |
| powder | | with faint | with faint | with faint | with faint | with faint |
| injection | | yellow color | yellow color | yellow color | yellow color | yellow color |
| | Content | 93.0 | 92.9 | 92.8 | 92.5 | 92.1 |
| | (%) | | | | | |

The result of the accelerated test is shown in Table 9. From the test, we can see that while under the status of lyophilized powder, the Forsythiaside lyophilized powder injection can have a good stability and its Description and content are not varied sufficiently which proves that it can comply with the requirement of injection preparation.

Next, we will take the water injection and the infusion solution as the example to clearly explain the reason for choosing the pharmaceutical adjuvant and the pH value in the preparation. And we will also specify the stability of the Forsythiaside water injection and the Forsythiaside infusion solution by accelerated test.

### 2.5 Choice of the Stabilizing Agents

The commonly used stabilizers in the water injection and infusion solution are Disodium EDTA, Sodium-Calcium EDTA, Calcium EDTA, Vitamin C and Pyrosulfite. We have designed an experiment for choosing a suitable type and a proper using amount of stabilizers. The result obtained is given set forth in Table 10.

TABLE 10
Experiment on the Choice of Stabilizing Agents of the
Forsythiaside Water Injection and Infusion Solution.

| | Using Amount | | |
|---|---|---|---|
| Type of the | □ Principal Agent: | | |
| stabilizer | Stabilizer□ | Product Appearance | Stability |
| Disodium EDTA | 1□ 0.2 | Clear liquid with faint | Fine |
| | | yellow color | |
| | 1□ 0.5 | Clear liquid with faint | Good |
| | | yellow color | |
| Sodium-Calcium | 1□ 0.5 | Clear liquid with faint | Fine |
| EDTA | | yellow color | |
| | 1□ 1 | Clear liquid with faint | Fine |
| | | yellow color | |
| Calcium EDTA | 1□ 0.3 | Clear liquid with faint | Fine |
| | | yellow color | |
| | 1□ 0.5 | Clear liquid with faint | Fine |
| | | yellow color | |
| Vitamin C | 1□ 2 | Clear liquid with faint | Good |
| | | yellow color | |
| | 1□ 5 | Clear liquid with faint | Fine |
| | | yellow color | |
| Pyrosulfite | 1□ 1 | Clear liquid with faint | Fine |
| | | yellow color | |
| | 1□ 3 | Clear liquid with faint | Good |
| | | yellow color | |

The result shows that: the water injection and the infusion solution can maintain stable when the proportion of Forsythiaside to stabilizing agent is 1: 0-5 by weight, and they can maintain stable better when the proportion is 1:0-3

### 2.6 Choice of the pH Value

Choice of the pH value of the water injection and the infusion solution is the same as that of the lyophilized powder injection. The pH value of the lyophilized powder injection in present invention is controlled at 3.0-6.0.

### 2.7 Accelerated Testing

Through accelerating medicine's chemical or physical changes, can we approach the stability of a pharmaceutical preparation. And it also provides necessary information in the fields of prescription design, technique improvement, quality research, package improvement, transportation and storage. The conditions that need to be observed are as follows:
Package: The package which is intended to be sold on the market (ampoule or infusion bottle)
Temperature: 40° C.±2° C.
Humidity: 75%±5%

TABLE 11
Result of the Accelerated Test

| | | Accelerated Test | | | | |
|---|---|---|---|---|---|---|
| Dosage Form | | 0 day | 15 days | 1 month | 2 months | 3 months |
| Water | Description | Clear liquid | Clear liquid | Clear liquid | Clear liquid | Clear liquid |
| Injection | | with faint | with faint | with faint | with faint | with faint |
| | | yellow color | yellow color | yellow color | yellow color | yellow color |
| | Content | 92.6 | 92.1 | 92.0 | 92.2 | 91.8 |
| | (%) | | | | | |
| Infusion | Description | Clear liquid | Clear liquid | Clear liquid | Clear liquid | Clear liquid |
| Solution | | with faint | with faint | with faint | with faint | with faint |
| | | yellow color | yellow color | yellow color | yellow color | yellow color |
| | Content | 92.2 | 92.0 | 91.9 | 92.0 | 91.5 |
| | (%) | | | | | |

The result of the accelerated test is shown in Table 11. From the test, we can see that while under the status of water injection and infusion solution, the Forsythiaside water injection can have a good stability and its Description and content are not varied sufficiently which proves that it can comply with the requirement of injection preparation.

1. A Forsythiaside injection preparation, prepared by Forsythiaside and pharmaceutical adjuvant with a proportion by weight of 1:(0-5).

2. The Forsythiaside injection preparation according to claim 1, wherein the proportion by weight of Forthiaside to pharmaceutical adjuvant is 1: (0-3).

3. The Forsythiaside injection preparation according to claim 1, wherein the injection preparation includes lyophilized powder injection, water injection and infusion solution.

4. The Forsythiaside injection preparation according to claim 3, wherein the pharmaceutical adjuvant of lyophilized powder injection is one or a mixture of any two of the following Mannitol, Glucose and Sorbitol.

5. The Forsythiaside injection preparation according to claim 4, wherein the proportion by weight of Forthiaside to pharmaceutical adjuvant is 1:0.

6. A preparative method for Forsythiaside injection preparation according to claim 4, wherein Forsythiaside and frame agent are mixed according to their proportion by weight, water for injection with 10˜50 times of the weight of the Forsythiaside is added to dissolve the mixture, then adjust the pH value to 3.0-6.0, fill it into ampoules after refined filtration and ultrafiltration. And the lyophilized injection is finally obtained after lyophilized according to the following lyophilizing curve.

| Temperature (° C.) | Time (h) |
|---|---|
| 0-−40 | 2 |
| −40 | 4 |
| −40-−25 | 3 |
| −25 | 5 |
| −25-30 | 2 |
| −30 | 9 |

7. The Forsythiaside injection preparation according to claim 3, wherein the pharmaceutical adjuvant used for the Forsythiaside water injection is one or several kinds of the following Disodium EDTA, Sodium-Calcium EDTA, Calcium EDTA, Vitamin C and Pyrosulfite.

8. A preparative method for Forsythiaside injection preparation according to claim 7, wherein it includes the following steps:
Adding pharmaceutical adjuvant into water for injection with its weight 0˜5 times of that of Forsythiaside, then stirring the solution until it dissolves completely;
Adding activated carbon with 0.5-0.05% weight of the total amount, stirring, filtering and decarbonizing;
Adding Forsythiaside to the filtrate and making it fully dissolved, regulating the pH value to 3.0-6.0;
Adding activated carbon with 0.2-0.02% weight of the total amount, stirring the solution at room temperature, filtering, decarbonizing;
Adding water for injection into the filtrate to maximum scale, measuring its pH value and the active constituent content. After being qualified, the injection should be filtered repeatedly to being clear, then filling it separately into ampoules, and have a sterilization and a package later.

9. The Forsythiaside injection preparation according to claim 3, wherein the pharmaceutical adjuvant used for Forsythiaside infusion solution is one or several kinds of the following Disodium EDTA, Sodium-Calcium EDTA, Calcium EDTA, Vitamin C and Pyrosulfite.

10. A preparative method for Forsythiaside injection preparation according to claim 9, including the following steps:
Adding Sodium Chloride or Glucose to water for injection and adding the pharmaceutical adjuvant with the adjuvant weight 0˜5 times of that of Forsythiaside, then stirring the solution until it dissolves completely;
Adding activated carbon with 0.5-0.05% weight of the total amount, stirring, filtering and decarbonizing;
Adding Forsythiaside to the filtrate and making it fully dissolved, regulating the pH value to 3.0-6.0;
Adding activated carbon with 0.2-0.02% weight of the total amount, stirring the solution at room temperature, filtering, decarbonizing;
Adding water for injection into the filtrate to maximum scale, measuring its pH value and the active constituent content. After being qualified, the injection should be filtered repeatedly to being clear, then filling it separately, sterilizing and packaging.

11. The Forsythiaside injection preparation according to claim 2, wherein the injection preparation includes lyophilized powder injection, water injection and infusion solution.

12. A preparative method for Forsythiaside injection preparation according to claim 5, wherein Forsythiaside and frame agent are mixed according to their proportion by weight, water for injection with 10˜50 times of the weight of the Forsythiaside is added to dissolve the mixture, then adjust the pH value to 3.0-6.0, fill it into ampoules after refined filtration and ultrafiltration. And the lyophilized injection is finally obtained after lyophilized according to the following lyophilizing curve.

| Temperature (° C.) | Time (h) |
|---|---|
| 0-−40 | 2 |
| −40 | 4 |
| −40-−25 | 3 |
| −25 | 5 |
| −25-30 | 2 |
| −30 | 9 |
